(19) United States
(12) Patent Application Publication
(10) Pub. No.: US 2008/0232655 A1
(43) Pub. Date: Sep. 25, 2008

(54) Apparatus and Method for Assisting the Diagnostic Evaluation of Images

(75) Inventors: Rafael Wiemker, Kisdorf (DE); Frans Andreas Gerritsen, Oisterwijk (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronic, N.V., Eindhoven (NL)

(21) Appl. No.: 10/595,802

(22) PCT Filed: Nov. 5, 2004
(86) PCT No.: PCT/IB04/52322

§ 371 Date: Jun. 3, 2008

(30) Foreign Application Priority Data
Nov. 13, 2003 (EP) 03104190.8

(51) Int. Cl. A61B 5/00; G06F 7/00

(52) U.S. Cl. 382/128; 707/104.1

(57) ABSTRACT

The invention relates to an apparatus and to a method for assisting a doctor in assessing a potentially pathological structure, such as a tumor (8) for example. In this connection, characteristic parameters (p₁⁰, p₂⁰, . . . ), such as its size or the edge contrast, are determined in a data-processing apparatus (1) from a three-dimensional image (7) of the structure (8). Furthermore, data records (4) with the characteristic parameters (p₁, p₂, . . . ) pertaining to documented examples, together with diagnostic additional information (q₁, q₂, . . . ), are stored in a database module (3). In the database module (3) it is then possible to establish those data records (4) that are similar to the structure (8) under consideration in respect of the characteristic parameters. These data records can be displayed on a monitor (5) and provide assistance to a doctor in his assessment of the current image.

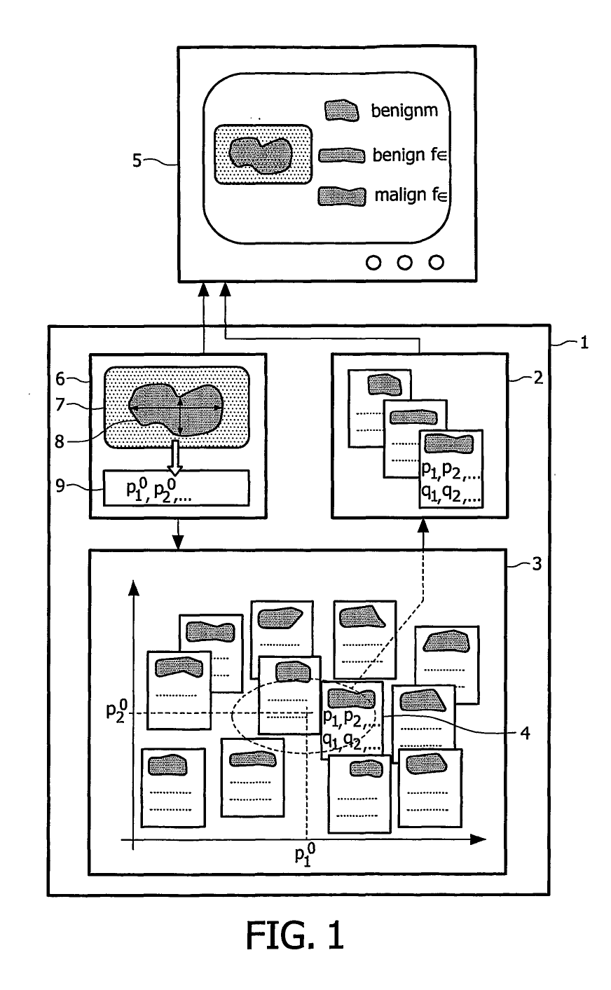

The invention relates to an apparatus and to a method for assisting the diagnostic evaluation of images of a potentially pathological structure, such as a tumor for example.

Modern imaging methods, such as X-ray computer tomography or magnetic resonance imaging (MRI), are to an increasing extent enabling high-quality two-dimensional and three-dimensional images to be produced from inside the body; on these images, potentially pathologically altered structures, such as tumors, for example, can be detected at an early stage. However, this presents the radiologists with the difficult task of carrying out a differential diagnosis of the typically small structure and having to plan further diagnostic or therapeutic steps. In this connection, it is known from U.S. Pat. No. 6,430,430 B1 to evaluate sectional views of the brain by means of automated image analysis methods in order to detect specific pathological alterations. Nevertheless, such methods afford no assistance for the differential diagnosis and for the decision on the further treatment of the case.

Against this background, it was an object of the present invention to provide means for assisting the diagnostic evaluation of images of a potentially pathological structure.

This object is achieved by an apparatus having the features of claim 1 as well as by a method having the features of claim 10. Advantageous embodiments are contained in the dependent claims.

The apparatus according to the invention is used for assisting the diagnostic evaluation of images from inside the body of a patient that show a potentially pathological structure. The images may be—preferably three-dimensional—X-ray images, MRI images or ultrasound images, for example. The potentially pathological structure may in principle be any body tissue or body organ that comes into consideration here, especially a potential tumor. The apparatus comprises the following components:

a) An analysis module, with which a set of characteristic parameters can be established from a given image of the potentially pathological structure, the parameters being related to the structure. The analysis module can be implemented, for example, in the form of a software program on data-processing equipment. The characteristic parameters can be, in particular, geometric characteristics of the structure and its surrounding area that are derivable from the image, as well as additional information relating to the imaged body area and to the patient.
(b) A database module, which contains stored data records relating to structures of the same kind (and, if applicable, data records of other structures). Each of the data records comprises here a medical example which pertains to the structure and is documented with its associated structure-related characteristic parameters (as per step a)) as well as together with additional information. The additional information can comprise, in particular, diagnostic results of the relevant example, that is, for example, biopsy results or disease courses. For a predetermined set of characteristic parameters the database module is furthermore designed to ascertain from the memory those data records whose associated characteristic parameters lie close to the predetermined set of characteristic parameters.
c) An output module, with which the data records ascertained in step b) can be processed further, for example, can be read out on a display device.

The described apparatus can substantially assist a doctor in his diagnostic assessment of a potentially pathological structure, by selecting and displaying to the doctor from a stored supply of documented examples those data records that have a similar appearance. The similarity of the examples is measured here by way of the characteristic parameters established with the analysis module. In this context it is important for the doctor that the established examples contain additional information that documents, for example, the further course of the disease or the results of further diagnostic steps. The doctor can therefore quickly get an idea of how typical the course of the disease is in the case of the structure being observed.

The structure-related parameters, which are established by the analysis module, for example, (partly) by means of automated image-processing methods and which are stored together with each data record, can preferably be at least one of the following variables: the volume of the structure, which is determinable in particular in the case of three-dimensional images; the compactness, the eccentricity, the needle-shape, the edge contrast, the homogeneity and/or opacity distribution of the structure; the texture of the tissue surrounding the structure (parenchyma); the number of blood vessels to which the structure is connected; the body region in which the structure is located (brain, lung, intestine, etc.); patient-related data, such as age, sex or risk group of the patient. A kind of fingerprint of the structure can be created from the selected variables, although it is not necessary to know in detail what variable is of crucial importance for classification of the structure and for what reasons.

In order to be able to compare the characteristic parameters of a structure under consideration with stored data records, a metric on the (multi-dimensional) area of the parameters is preferably defined. In this connection, advantageously as much a priori information as possible about the function of the characteristic parameters is implemented, so that the results determined with the metric have the greatest possible relevance. If, for example, it is known that the edge contrast of a structure is of considerable importance for the assessment of a tumor, the metric can weight these parameters correspondingly strongly. Furthermore, the metric can be nonlinear, especially nontranslation-invariant. For example, by varying the metric with the parameter “body region”, one can take into account the fact that, depending on the body region (lung, intestine etc.), specific characteristic parameters of a suspect structure can be accorded different significance.

According to a further aspect of the invention, images of the respective associated structure are also contained in the data records of the database module. These images allow the doctor to draw a direct visual comparison between the structure currently under observation and the data records established for that purpose.

The apparatus additionally comprises a display device, such as a monitor, for example, for displaying the image of the structure and/or for displaying information from established data records.

According to another aspect of the apparatus, this contains an input means, such as a keyboard or a mouse, by means of which an interactive analysis of the structure in the analysis module is possible. In this way, for example, a doctor can mark the center or the edges of a suspect structure on an image, whereupon the analysis module then automatically determines further parameters of this structure.

The invention relates furthermore to a method for assisting the diagnostic evaluation of images of a potentially pathological structure, which method comprises the following steps:

a) setting up a database, which contains data records that relate to structures of the same kind and document respective examples with associated characteristic parameters of the structure plus additional information;
b) establishing a set of characteristic parameters that are related to the structure portrayed in the image;
c) establishing and preferably displaying data records from the database, the associated characteristic parameters of which data records lie close to the set of characteristic parameters established in step b).

The method comprises in general form the steps that can be performed with an apparatus of the kind described above. As regards the details, advantages and further aspects of the method, the reader is therefore referred to the above description.

These and other aspects of the invention are apparent from and will be elucidated, by way of non-limitative example, with reference to the embodiment(s) described hereinafter.

In the drawings:

The single FIGURE shows schematically an apparatus according to the invention for assisting the diagnostic evaluation of an image.

The apparatus is formed substantially by a data-processing unit 1 (workstation), which in its turn contains various modules. These modules are typically implemented by (electronic) memories or memory contents as well as associated programs (software).

From an imaging device, not shown more specifically, such as an X-ray CT or an MRI apparatus, a three-dimensional image 7 in the form of a corresponding dataset is provided for the data-processing unit 1. In this image, which can be, for example, a picture of the lung or the intestine, there is a suspect structure 8 or structure identified as a tumor. In such a situation, the radiologist in charge has to make a differential diagnosis of the findings and decide on further diagnostic or therapeutic steps. Through the method described below, he is assisted in this by the data-processing unit 1.

As regards the method, first of all the image 7 of the tumor 8 is analyzed fully or semi-automatically in an analysis module 6 of the data-processing unit 1. In a semi-automatic analysis, by means of a computer mouse (not shown) the radiologist selects, for example, the region of the tumor 8 of interest to him. The tumor is then automatically three-dimensionally segmented by the analysis module 6 using known algorithms of image-processing. The analysis module 6 furthermore determines quantitatively a set 9 of characteristic parameters p₁⁰, p₂⁰, . . . of the tumor, for example, its volume, its compactness, its eccentricity, its needle-shape, its edge contrast, its homogeneity, its opacity distribution, the structure of the surrounding parenchyma, the number of blood vessels supplying the tumor, and so on. Furthermore, associated background information, such as the imaged body region, as well as sex, age and risk group of the patient are determined. From all the information mentioned, the analysis module 6 produces a set 9 of parameters characteristic of the tumor 8.

The data-processing unit 1 additionally contains a database module 3, in which a large number of documented tumor data records are stored. Each data record 4 contains here a (three-dimensional) image of the relevant tumor and its surrounding tissue as well as the associated set of characteristic parameters, which are defined as described above for the analysis module. Moreover, each data record 4 contains additional information that is not (yet) present in the case of the tumor 8 currently being examined. This additional information comprises, for example, the confirmed differential diagnosis of the documented tumor, its biopsy result, the further course of the disease, and the like.

The set 9 of characteristic parameters p₁⁰, p₂⁰, . . . of the tumor 8 currently under consideration determined in the analysis module 6 is transmitted to the data-base module 3, whereupon this ascertains in the database those data records 4 of documented examples whose associated characteristic parameters lie close to the supplied set 9 (dotted circle in the FIGURE). The spacing of sets of the characteristic parameters is determined by means of a metric of the parameter area. This metric is defined in suitable manner, so that it determines similarities and dissimilarities of tumors in relation to their clinical importance. The metric can here be differently defined in particular for different types of tumors or for different body regions. The metric can be defined, for example, by means of the Euclidean spacing in the multi-dimensional parameter area, the different axes each being (locally) weighted differently.

A suitable number of, for example, ten examples 4, which were ascertained by the database as being similar to the current tumor 8, is finally passed to an output module 2 (only three of these data records are illustrated in the FIGURE). The output module 2 can display the images belonging to these data records on a monitor 5, together with the (additional) information of interest, such as the differential diagnosis and the sex of the patient in question for example. The image of the tumor 8 currently being examined is preferably displayed on the monitor 5 at the same time, so that the radiologist is able to make a direct visual comparison. Furthermore, he can easily ascertain whether in the majority of established similar cases for example benign or malignant tumors were diagnosed and what the respective further course of the disease was. This considerably assists the doctor in his diagnosis and in his decision about the further course of action (biopsy, follow-up, resection etc.), although the system itself makes neither evaluations nor suggestions and hence does not prejudice independent medical decision.

1. An apparatus (1) for assisting the diagnostic evaluation of images (7) of a potentially pathological structure (8), containing:
a) an analysis module (6), with which a set (9) of characteristic, structure-related parameters (p₁⁰, p₂⁰, . . . ) can be established from an image (7) of the structure (8);
b) a database module (3), which contains stored data records (4) relating to structures of the same kind, which data records document respective examples with their associated characteristic parameters (p₁, p₂, . . . ) and with additional information (q₁, q₂, . . . ) wherein for a predetermined set (9) of characteristic parameters (p₁⁰, p₂⁰, . . . ) the database module (3) is able to establish those of said data records (4) whose characteristic parameters (p₁, p₂, . . . ) lie close to the predetermined set (9);
c) an output module (2), for further processing of the established data records (4).

2. An apparatus as claimed in claim 1, characterized in that the images (7) of the structure (8) are X-ray images, MRI images or ultrasound images.

3. An apparatus as claimed in claim 1, characterized in that the structure (8) is a potential tumor.

4. An apparatus as claimed in claim 1, characterized in that the parameters (p₁, p₂, . . . ; p₁⁰, p₂⁰, . . . ) related to the structure (8) comprise at least one of the following variables: volume, compactness, eccentricity, needle-shape, edge contrast, homogeneity, opacity distribution, texture of the surrounding parenchyma, number of supplying blood vessels, body region being imaged, as well as patient-related data such as age, sex and/or risk group.

5. An apparatus as claimed in claim 1, characterized in that the additional information (q₁, q₂, . . . ) comprises diagnostic results, especially biopsies, and/or disease courses.

6. An apparatus as claimed in claim 1, characterized in that a metric is defined on the area of the characteristic parameters (p₁, p₂, . . . ; p₁⁰, p₂⁰, . . . ).

7. An apparatus as claimed in claim 1, characterized in that the data records (4) comprise at least one image of the associated structure.

8. An apparatus as claimed in claim 1, characterized in that it contains a display device (5) for displaying an image (7) of the structure (8) and/or information from data records (4).

9. An apparatus as claimed in claim 1, characterized in that it contains input means for an interactive analysis of the image (7) of the structure in the analysis module (6).

10. A method for assisting the diagnostic evaluation of images (7) of a potentially pathological structure (8), comprising the following steps:
a) setting up a database (3), which contains data records (4) that relate to such a structure and document respective examples with associated characteristic parameters (p₁, p₂, . . . ) of the structure plus additional information (q₁, q₂, . . . );
b) establishing a set (9) of characteristic parameters (p₁⁰, p₂⁰, . . . ) that are related to the potentially pathological structure (8);
c) establishing data records (4) from the database (3), the accompanying characteristic parameters (p₁, p₂, . . . ) of which data records lie close to the set (9) of parameters established in step b).
